(19) United States
(12) Patent Application Publication
(10) Pub. No.: US 2026/0054021 A1
(43) Pub. Date: Feb. 26, 2026

(54) IMPROVEMENTS TO AUTOMATIC POSITIVE AIRWAY PRESSURE MACHINES

(71) Applicant: HALARE, INC., Sewickley, PA (US)

(72) Inventor: Anthony C. Warren, Wien (AT)

(73) Assignee: HALARE, INC., Sewickley, PA (US)

(21) Appl. No.: 19/102,633

(22) PCT Filed: Feb. 10, 2023
(86) PCT No.: PCT/US2023/012772

§ 371 Date: Feb. 10, 2025

Related U.S. Application Data
(60) Provisional application No. 63/396,714, filed on Aug. 10, 2022.

(51) Int. Cl. A61M 16/00; A61M 16/06

(52) U.S. Cl. CPC A61M 16/024; A61M 16/0069; A61M 16/06; A61M 2016/0027; A61M 2016/003; A61M 2230/06; A61M 2230/205

(57) ABSTRACT

A positive airway pressure (PAP) device including an air pump for blowing air into a mask worn by a user, a sensor for detecting air pressure or air flow-rate produced by the air pump, a communication interface for communicating with a physiological parameter sensor worn by the user for detecting a physiological parameter of the user, and a processor. The processor is configured to control the air pump to blow the air into the mask according to the air pressure or air flow-rate sensed by the sensor to achieve a prescribed air pressure or air flow-rate, and control the air pump to adjust the prescribed air pressure or air flow-rate in response to the detected physiological parameter.

### TECHNICAL FIELD

The present disclosure relates to systems, methods, and apparatuses for aiding patients to alleviate symptoms of sleep disordered breathing (SDB) including but not limited to sleep apnea, hypopnea, snoring and atrial fibrillation.

### BACKGROUND

A significant proportion of Sleep Disordered Breathing (SDB) relates to a condition characterized by repeated episodes of hypopnea (underbreathing) and apnea (not breathing) during sleep, usually resulting in reduction in blood oxygen saturation (SpO₂), and related changes in heartrate, blood pressure and arousal from sleep and sympathetic nervous system activation. Sometimes SDB causes snoring which may or may not be associated with episodes of hypopnea (under-breathing) and apnea (not breathing). Further, related sympathetic nervous system activation may trigger unstable heart patterns known as atrial fibrillation. These episodes are referred to as events throughout this disclosure.

Events may last 10-20 seconds or more and can occur 20 to 30 times or more an hour. The most common type of SDB affecting approximately 85% of patients is referred to as Obstructive Sleep Apnea (OSA) in which physical obstruction of the airways occurs due to sleep related loss of upper airway dilator muscle tone. A preferred treatment for OSA is the use of a Positive Air Pressure devices (PAP). PAP's occur in several different configurations including a continuous positive airway pressure (CPAP) device, an automatic positive airway pressure (APAP) device, or a Bi-Level positive airway pressure (BiPAP) device.

A CPAP device requires a patient to wear a mask that delivers a prescribed air pressure/flow-rate during sleep in order to keep the airways to the lungs open. One improvement to CPAP is Automatic Positive Airway Pressure (APAP). While a CPAP has continuous pre-set pressure/flow-rate setting(s), an APAP is able to modify applied airway pressure/flow-rate by measuring how much resistance is present while breathing causing restriction in airway airflow to the lungs and/or measuring the airflow to the lungs. The control algorithms in an APAP machine allow it to remain on a low pressure/flow-rate setting until a change in breathing is detected and more airflow is required. Although an improvement on CPAP devices, current APAPs have certain shortcomings which are addressed by the disclosures herein. Furthermore, the disclosure described herein adds additional functionality to PAP devices including detection and alleviation of SDB events such as AF and snoring episodes (SE).

### SUMMARY

One example embodiment includes a positive airway pressure (PAP) device including an air pump for blowing air into a mask worn by a user, a sensor for detecting air pressure or air flow-rate produced by the air pump, a communication interface for communicating with a physiological parameter sensor worn by the user for detecting a physiological parameter of the user, and a processor. The processor is configured to control the air pump to blow the air into the mask according to the air pressure or air flow-rate sensed by the sensor to achieve a prescribed air pressure or air flow-rate, and control the air pump to adjust the prescribed air pressure or air flow-rate in response to the detected physiological parameter.

In an example embodiment the PAP device is one of a continuous positive airway pressure (CPAP) device, an automatic positive airway pressure (APAP) device, or a Bi-Level positive airway pressure (BiPAP) device.

In an example embodiment the physiological parameter sensor is worn by the user separate from the mask.

In an example embodiment the physiological parameter sensor is integrated into the mask or the PAP device.

In an example embodiment the physiological parameter sensor is a blood oxygen sensor and the processor is configured to monitor blood oxygen levels of the user over time, and control the air pump to increase the prescribed air pressure or air flow-rate in response to the blood oxygen levels decreasing below a blood oxygen level threshold.

In an example embodiment the physiological parameter sensor is a heart rate sensor and the processor is configured to monitor heart rate patterns of the user over time, and control the air pump to increase the prescribed air pressure or air flow-rate in response to determining that the heart rate patterns are abnormal.

In an example embodiment the physiological parameter sensor is a blood pressure sensor and the processor is configured to monitor blood pressure of the user over time, and control the air pump to increase the prescribed air pressure or air flow-rate in response to the blood pressure increasing above a blood pressure threshold.

In an example embodiment the processor is configured to control the air pump to adjust the prescribed air pressure or air flow-rate in response to a pressure event indicated by the pressure sensor, or a physiological event indicated by the detected physiological parameter.

In an example embodiment the processor is configured to control the air pump to adjust the prescribed air pressure or air flow-rate in response to the detected physiological parameter being compared to a physiological parameter threshold or a baseline of the user.

In an example embodiment the processor is configured to switch between controlling the air pump based on the detected air pressure or air flow-rate and the detected physiological parameter.

One example embodiment includes a positive airway pressure (PAP) device including air pump for blowing air into a mask worn by a user, a communication interface for communicating with a physiological parameter sensor worn by the user for detecting a physiological parameter of the user, and a processor configured to control the air pump to adjust the prescribed air pressure or air flow-rate in response to the detected physiological parameter.

In an example embodiment the physiological parameter sensor is worn by the user separate from the mask.

In an example embodiment the physiological parameter sensor is integrated into the mask or the PAP device.

In an example embodiment the physiological parameter sensor is a blood oxygen sensor and the processor is configured to monitor blood oxygen levels of the user over time, and control the air pump to increase the prescribed air pressure or air flow-rate in response to the blood oxygen levels decreasing below a blood oxygen level threshold.

In an example embodiment the physiological parameter sensor is a heart rate sensor and the processor is configured to monitor heart rate patterns of the user over time, and control the air pump to increase the prescribed air pressure or air flow-rate in response to determining that the heart rate patterns are abnormal.

One example embodiment includes a method for controlling a positive airway pressure (PAP) device. The method includes blowing air, by an air pump, into a mask worn by a user, communicating, by a communication interface, with a physiological parameter sensor worn by the user for detecting a physiological parameter of the user, and controlling, by a processor, the air pump to adjust the prescribed air pressure or air flow-rate in response to the detected physiological parameter.

In an example embodiment the method includes monitoring, by the physiological parameter sensor, blood oxygen levels of the user over time, and controlling, by the processor, the air pump to increase the prescribed air pressure or air flow-rate in response to the blood oxygen levels decreasing below a blood oxygen level threshold.

In an example embodiment the method includes monitoring, by the sensor, heart rate patterns of the user over time, and controlling, by the processor, the air pump to increase the prescribed air pressure or air flow-rate in response to determining that the heart rate patterns are abnormal.

In an example embodiment the method includes increasing, by the processor, the prescribed air pressure or air flow-rate in response to the detected physiological parameter indicating onset of at least one of a heart rate abnormality, a low SpO2, or snoring.

In an example embodiment the method includes maintaining or decreasing, by the processor, the prescribed air pressure or air flow-rate in response to the detected physiological parameter indicating an absence of at least one of a heart rate abnormality, a low SpO2, or snoring.

### BRIEF DESCRIPTION OF DRAWINGS

FIG. 1A shows a bar graph of the correlation between snoring severity and plaque formation in the carotid arteries, according to an example embodiment of the disclosure.

FIG. 1B shows a bar graph of the correlation between severity of sleep apnea and occurrence of atrial fibrillation, according to an example embodiment of the disclosure.

FIG. 2 shows a data plot of the changes in SpO₂, Heartrate, Blood Pressure and Atrial Fibrillation during a typical SDB event in which AF is triggered by central nervous system arousal, according to an example embodiment of the disclosure.

FIG. 3A shows a data plot illustrating an APAP device detecting low flow-rate to gradually increase pressure/flow-rate until flow rate normalizes, according to an example embodiment of the disclosure.

FIG. 3B shows a data plot on how data from an independent blood oxygen concentration sensor correlates with the pressure/flow-rate data recorded in FIG. 3A, according to an example embodiment of the disclosure.

FIG. 4 shows data plots of an example of an APAP device where the airflow rate sensor does not detect apnea and hypopnea events and thereby fails to provide therapeutic benefit, according to an example embodiment of the disclosure.

FIG. 5 shows pulse traces showing a comparison between a normal PPG waveform and a hypoxic event PPG waveform, according to an example embodiment of the disclosure.

FIG. 6 compares a normal PPG trace and another PPG trace exhibiting AF, according to an example embodiment of the disclosure.

FIG. 7A shows a flowchart of APAP pressure/flow-rate control using indirect airway patency sensing, according to an example embodiment of the disclosure.

FIG. 7B shows a flowchart of APAP pressure/flow-rate control using a combination of indirect airway patency sensing and direct sensing of heart rhythm, according to an example embodiment of the disclosure.

FIG. 7C shows a flowchart of APAP pressure/flow-rate control using a combination of indirect airway patency sensing and direct sensing of snoring, according to an example embodiment of the disclosure.

FIG. 7D shows a flowchart of APAP pressure/flow-rate control using a combination of indirect airway patency sensing and direct sensing of SpO2, according to an example embodiment of the disclosure.

FIG. 7E shows a flowchart of APAP pressure/flow-rate control using a combination of indirect airway patency sensing and direct sensing of both heart rhythm and SpO2, according to an example embodiment of the disclosure.

FIG. 7F shows a flowchart of APAP pressure/flow-rate control using a combination of indirect airway patency sensing and direct sensing of blood pressure, according to an example embodiment of the disclosure.

FIG. 7G shows a flowchart of APAP pressure/flow-rate control using a combination of indirect airway patency sensing and direct sensing of heart rhythm, SpO2 and blood pressure, according to an example embodiment of the disclosure.

FIG. 7H shows a flowchart of APAP pressure/flow-rate control using direct sensing of SpO2, according to an example embodiment of the disclosure.

FIG. 8 is an example of a decision matrix for modifying APAP applied pressure/flow-rate, according to an example embodiment of the disclosure.

FIG. 9 shows a wearable ring able to sense both blood oxygen concentration and heartrate and communicate wirelessly to an APAP to control applied airway pressure/flow-rate, according to an example embodiment of the disclosure.

FIG. 10 shows a smartphone able to sense snoring and communicate wirelessly to an APAP to control applied airway pressure/flow-rate, according to an example embodiment of the disclosure.

FIG. 11 shows a mask with a built-in sensor that communicates wirelessly to an APAP to control applied airway pressure/flow-rate, according to an example embodiment of the disclosure.

### DETAILED DESCRIPTION

This disclosure relates to systems, methods, and apparatuses for aiding patients suffering from sleep disordered breathing (SDB) including sleep apnea and hypopnea, with or without co-existing atrial fibrillation (AF) episodes by improving the performance of Positive Airway Pressure (PAP) machines. Specifically, one or more sensors detect ‘direct’ cardio-pulmonary physiological parameters of the user such as, but not limited to heart rate (HR), heartrate variability (HV), blood oxygen concentration level (Sp0₂,), blood pressure (BP) and the partial pressure of carbon dioxide (PCO₂) which are used to improve airway pressure control algorithms. Additionally, for patients suffering from snoring episodes, a sound sensor may be used to control the PAP device.

In one example, this disclosure describes a PAP device having a mask worn by a sufferer of SDB for the alleviation of apnea or hypopnea events, associated AF events and snoring episodes (SE) that varies the applied pressure/flow-rate to an airway. Control of the applied pressure/flow-rate is derived partly or fully on time variations of one or more physiological parameters of the sufferer. Such parameters may include, but are not limited to the cardio-pulmonary physiological parameters of the user mentioned above. Again, these parameters are referred to as ‘direct’ parameters, in contrast with ‘indirect’ parameters such as airway pressure/flow-rate used to control state of the art APAPs. Using direct parameters to determine necessary APAP pressure/flow-rate changes is beneficial because they are related directly, rather than indirectly, to the dangerous health impacts that SDB, AF and SE can impose on a sufferer. Indirect parameters currently used in APAP devices are, in some cases, inadequate to detect potentially dangerous physiological damage occurring in real-time and thereby: a) do not provide the therapeutic benefits required to fully and accurately alleviate SDB, AF symptoms and SE, and b) may exacerbate both short and long-term serious health problems.

Direct physiological parameters values are detected using one or more physiological parameter sensors connected to the PAP device directly or wirelessly. One or more physiological parameter sensors are worn by or placed proximate to a sufferer of SDB including but not limited to the user's face, an earlobe, an ear-canal, a wrist, a fingertip or a finger-stem. Sensors may also be embedded in or physically attached to the PAP device or mask.

Sensors sample the direct parameters such as HR, HV, SpO₂, BP and snoring sounds continuously or periodically such that events may be detected/predicted and appropriate changes to APAP pressure/flow-rate may be made promptly to alleviate or eliminate SDB, AF and SE. One or more of these direct physiological parameters may be used solely for the control of APAP applied pressure/flow-rate or in combination with other sensed indirect data such as airway resistance or airway flowrate. These data are inputted into a processor (not shown) which uses pre-programmed decision matrices to modify the applied airway pressure/flow-rate. The data for multiple sleep sessions are stored and machine learning algorithms are used to modify the decision matrices depending on personal response data over time and to predict the onset of events so that pressure/flow-rate changes can be invoked prior to events to minimize their severity. Although not shown, the processor may be one or more processors included in the PAP device itself, the user's smartphone or a backend server that may be in communication with the PAP device and/or the smartphone.

Improving the performance of APAP devices is beneficial in order to: a) provide more effective therapy for SDB, b) reduce or alleviate periods of AF and snoring that may occur during sleep, commonly associated with SDB events, and c) ensure that both short and long-term potentially dangerous health problems are alleviated. These benefits are made by sensing the key cardio-pulmonary parameters as well as sound sensors rather than solely relying on indirect airway pressure/flow-rate which alone may not be directly related to therapeutic requirements. Conventional PAPs that rely solely on airway patency detection have been shown to be inadequate in responding to some events in practice.

In general, SDB can have major short-term and long-term deleterious health related impacts. When sleep is interrupted throughout the night, drowsiness occurs during the day. People with SDB have twice the risk for car accidents, are 25% more likely to have at-work accidents and exhibit loss of work efficiency. SDB may also lead to long-term serious, chronic health issues, such as increased chance of stroke and other cardio-vascular diseases, and dementia. Roughly 38,000 cardiovascular deaths annually are in some way related to SDB.

SDB is often un-diagnosed in many cases until symptoms have become life threatening. Estimates are between twelve and twenty million Americans alone suffer from SDB. The economic impact of SDB in the US alone is estimated to be several billion dollars annually, which does not take into account the cost of long-term care associated with other chronic diseases resulting from SDB.

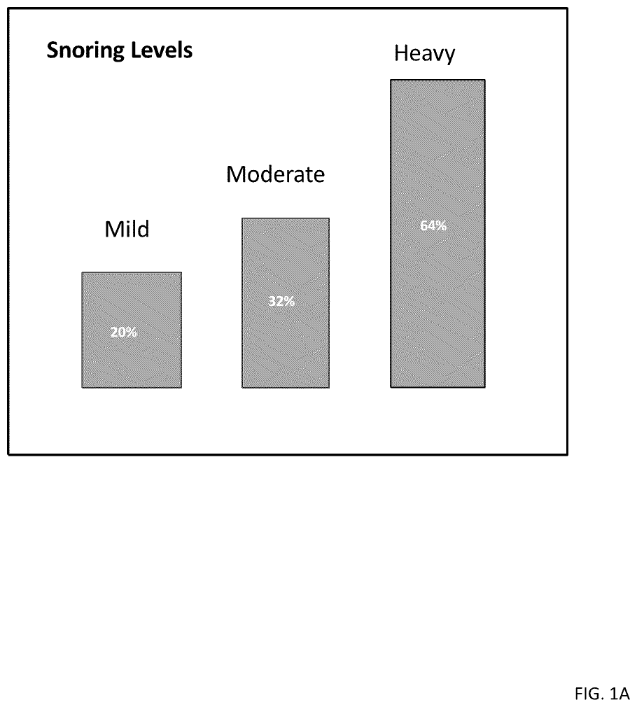

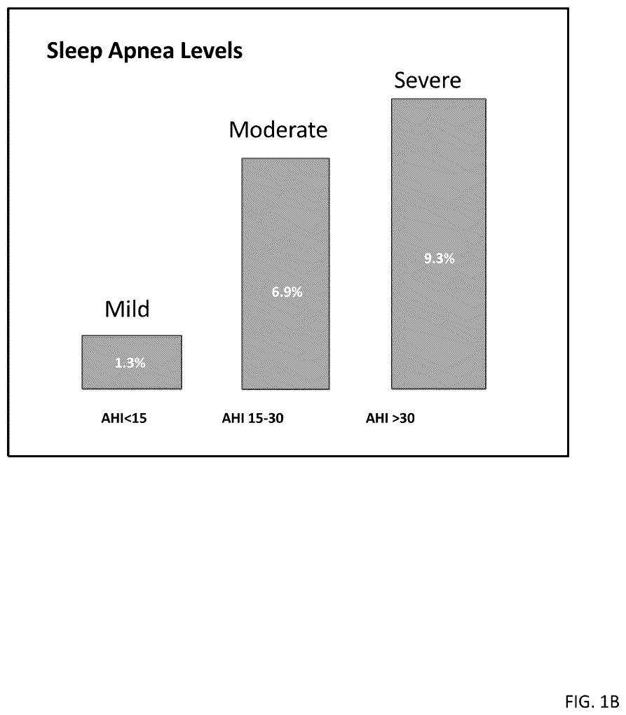

One common form of SDB, snoring, is a health hazard not only for the snorer but for their sleep partner and for their relationship. Snoring has been shown to directly lead to a stroke. Vibrations in the upper airways occurring during snoring trigger the formation of blood-flow-reducing plaque in the carotid arteries that carry oxygen-rich blood directly from the heart to the brain. This dangerous plaque formation depends on the intensity of snoring. Compared with non-snorers, carotid artery constriction has been found in 20% of mild snorers, 32% in moderate snorers and a disturbing 64% in heavy snorers. In these investigations, higher risks were there whether the snorers had sleep apnea or not. The correlation between snoring intensity and carotid artery plaque formation is shown in FIG. 1A.

According to the US National Sleep Foundation as many as 25% of married couples sleep in separate beds and 10% have separate bedrooms. The main reason is that 41% of sleep partners snore. In addition, sleep partners of snorers lose at least one hour of quality sleep every night. This deficit can lead to major long-term health problems, as well as a heightened chance of serious car and work accidents, irritability at work and in the home. Snoring is estimated to be the third most common cause of divorce in the United States and Great Britain. The disclosure herein provides a direct way of detecting and alleviating snoring both when associated with other SDB symptoms and when occurring alone.

Atrial fibrillation (AF) refers to an irregular heartbeat pattern that begins in the upper chambers of the heart (atria) when the normal cycle of electrical impulses in the heart is disturbed. This leads to a fast, chaotic heart rhythm and poor movement of blood from the atria to the heart's lower chambers (ventricles). AF, if untreated, can lead to a stroke and other serious medical complications. To make matters more complicated, AF is often asymptomatic, and it may remain undiagnosed until or even after development of complications, such as stroke. Therefore, early detection of AF is important.

AF is known to be correlated with SDB. FIG. 1B shows the magnitude of this relationship. The severity of sleep apnea is designated by the index AHI, defined later in this disclosure. The greater the severity of sleep apnea, the more likely a sufferer of SDB will experience AF. When sleep apnea is ranked as severe, close to 10% of sufferers will experience some symptoms of AF. Not all SDB sufferers however show AF symptoms. AF triggered by SDB events may however develop over time, and a benefit of the disclosure herein is the ability to detect when and if AF symptoms develop during therapeutic treatment for SDB.

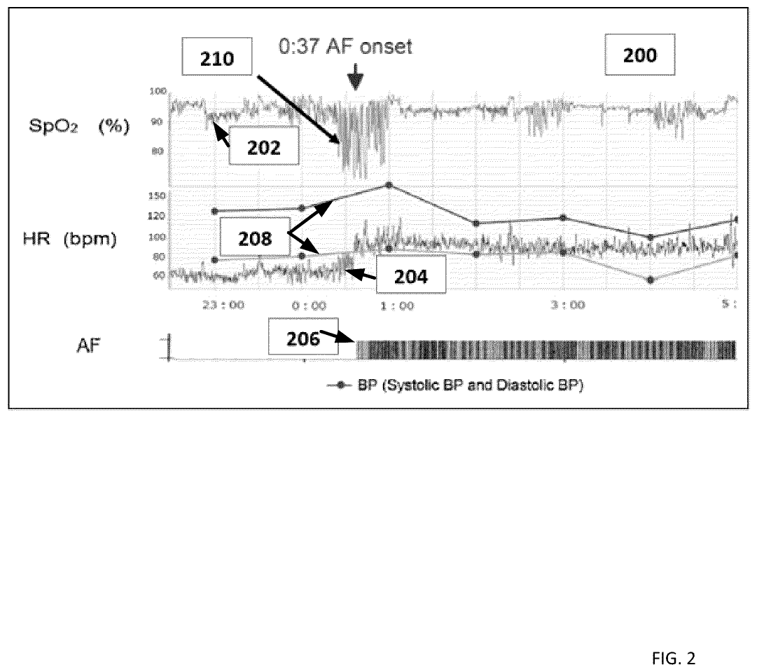

The onset of AF during sleep is often triggered by an SDB event. Chart 200 in FIG. 2 illustrates an SDB event in which several direct cardio-pulmonary parameters are monitored simultaneously, namely SpO₂ 202, heart rate 204, AF 206, and Systolic and Diastolic components of blood pressure 208. In this example, the first indication of the occurrence of an SDB event is the decrease in SpO₂, 210 at approximate time, 0.3 minutes. Not until later in the event do other cardio-pulmonary parameters show changes. Specifically, heart rate 204 and blood pressure, 208 increase at a later time. When SpO₂ has returned to normal values, blood pressure gradually returns to normal levels. But in this example, the event has triggered onset of AF at time 0.37 minutes, and the AF once triggered continues in 206 for well beyond the SDB event with the heartrate now unstable and at a high rate.

These deleterious effects arise principally because, when apnea and hypopnea events occur during sleep and airflow to the lungs is stopped or restricted, the concentration of oxygen in the blood declines to a dangerous level starving key organs such as the brain and heart from vital oxygen. Repeated periodic changes in SpO₂ usually result in concurrent changes in heart rate, which increases in an attempt to compensate for a drop in blood oxygen and thereby maintain sufficient oxygen supply to vital organs before returning to a baseline value once the SpO₂ value has returned to a pre-event value. Furthermore, this reduction in oxygen concentration is believed to trigger an arousal state in the central nervous system, which in turn, can prompt the heart rate to rise and in some cases result in extended periods of AF. Repeated oxygen depletion often coupled with variations in heart rate resulting from SDB events can result in both short-term and long-term chronic health problems, including stroke and early death.

There are a variety of treatments for SDB and sleep-related AF. These include lifestyle changes, special pillows or devices to avoid sleeping on the back, oral appliances to keep the airways open, methods for training tongue muscles, and breath training which neuro-plastically modifies the autonomous breathing control neural networks in the brain. If these are inadequate, continuous positive airway pressure devices (CPAP), the most prevalent type of Positive Airway Pressure devices (PAP), are prescribed. One class of PAP devices is referred to as APAP. Rather than using a fixed pressure/flow-rate to maintain the airways open as in a CPAP device, the device measures the resistance to airflow into the lungs and/or the flow of air into the lungs and increases the pressure/flow-rate when the flow rate and/or resistance to flow rate fall below certain pre-determined values for pre-determined time periods

The seriousness of SDB and OSA historically has been classified using standard definitions and indices as follows. These are derived from the detection of a series of hypopnea or apnea events during sleep. According to one standard, a hypopnea event is defined as a reduction in airway airflow by at least 50% for at least 10 seconds, while an apnea event is defined as a reduction in airway airflow by at least 75% for at least ten seconds.

The most commonly used index to classify the level of SDB is the Apnea-Hypopnea Index, (AHI). This is the number of events recorded per hour of sleep. There is increasing concern that AHI is based on indirect factors and not on direct physiological parameters. Nevertheless, for historical reasons it is still the most prevalent index. Based on AHI, the severity of OSA is classified as follows on number of events per hour irrespective of severity:
None/Minimal: AHI<5 per hour
Mild: AHI≥5, but <15 per hour
Moderate: AHI≥15, but <30 per hour
Severe: AHI≥30 per hour

A second index for classifying the level of SDB, is calculated from detected reductions in blood oxygen levels SpO₂ (desaturations) which often occur during SDB events. A drop in this parameter is directly related to health problems. At sea level, a normal blood oxygen level (saturation) is 96-97%. Reductions to not less than 90% usually are considered mild. Dips into the 80-89% range are considered moderate, and those below 80% are severe. The oxygen desaturation index (ODI) can be used to evaluate the severity of sleep apnea. ODI is defined as the number of events of oxygen desaturation per hour of sleep, with oxygen desaturation defined as a decrease in blood oxygen saturation (SpO₂) to lower than 3% below an established baseline value. Sometimes a drop of 6% below an established baseline is used. ODI records indicate whether the 3% or 6% standard apply.
Based on the ODI, the severity of OSA may be classified as follows:
None/Minimal: ODI <5 % decrease over defined time
Mild: ODI ≥5, but <15% decrease over defined time
Moderate: ODI≥15, but <30% decrease over defined time
Severe: ODI≥30 % decrease over defined time.

Creating an index to grade snoring intensity is complicated by the fact that snoring may occur with and without different levels of OSA, while individual snoring patterns differ significantly in terms of volume, frequency spectrum, length of snoring episodes, number of episodes per night and whether snoring occurs on a regular basis.

Snoring is generally characterized in three broad categories. At the lowest level, for example, if an individual snores once in a while, and not very loudly and breathing remains normal, then the classification is mild and potential health problems associated with this snoring are minimal. One negative outcome may be if the sleep partner is a light sleeper or is disturbed by the snores. This may cause personal disagreements between the partners, in which case treatment may be advised.

At the next level, individuals exhibit snoring behavior on three or more days of every week. They may have some difficulty with breathing during sleep. This sleep-associated breathing problem may result in daytime sleepiness and fatigue. Treatment should be sought. This level may co-exist with apnea or hypopnea events.

At the highest level, the sleeper snores very loudly, so that the sound is heard outside the bedroom. In many cases, but not always, the individual suffers from obstructive sleep apnea (OSA). As a result, the airways are totally or partially obstructed for ten seconds or more on average. This causes the brain to shift from deep sleep to lighter stages prematurely, depriving the person of sufficient restful sleep. Many people with OSA wake up during this phase for a brief time, though they have no memory of the awakenings. Several such episodes occur during the night, which translates to acute sleepiness the next day and often falling asleep during normal conversation or work. This, of course, impairs performance and social functioning. Treatment, even without OSA symptoms is highly recommended.

In order to determine whether snoring is occurring together with OSA, the sound power spectrum obtained by recording both snore intensity and frequency simultaneously may be invoked. Simple snores usually peak at a low frequency of 100-300 Hz, in contrast to the OSA coupled snore which typically peaks above 1000 Hz. Filters may be applied to the snoring sound to determine whether the snoring is co-existing with OSA. Additionally, according to the disclosure herein, monitoring cardio-pulmonary parameters such as SpO2 may be used for such determinations.

Artificial Intelligence (AI) methods may be implemented to garner further information for controlling a PAP. Prior to subjecting the sound signals to the AI methods, pre-processing algorithms are used to remove background environmental sounds, sounds generated by a sleep partner, and sounds created by any movement by the patient. In some cases, it is suggested that sound monitoring is undertaken when sleeping alone in a quiet space. In addition, or instead of sound power analysis, machine learning (ML) methods using classifiers from multiple records of sound signals from diagnosed sufferers of snoring may be used in diagnosing different levels of snoring from such signals. Further so-called Deep learning (DL) models, which unlike ML, automatically learn feature representations from subject's own sound datasets, sparing the tedious task of feature crafting using ML methods. DL uses a neural network, a set of interconnected layers of computational nodes. The most common DL approaches, used for snoring detection, are based on Convolutional Neural Networks (CNN).

APAP devices detect changes in airflow and/or airway flow resistance to control the applied air pressure/flow-rate and therefore commonly use AHI rather than ODI as an indicator of effectiveness in reducing the severity of SDB and also related AF events. However, as AHI does not relate to important direct cardio-pulmonary factors, it is not the most appropriate index for tracking SDB severity

An APAP operates as follows. When the air flow is sufficient to maintain a regular healthy breathing pattern, the pressure is held at a pre-determined (low) value, say 5 centimeters (cm) of positive pressure above ambient. If the air flow falls as a result of one or more events, the air pressure/flow-rate is increased, for example in stages, until sufficient airflow is re-established. The PAP device usually has a defined pressure range within which the controls are confined. A range of between 5 cm and 15 cm of positive pressure above ambient is typical.

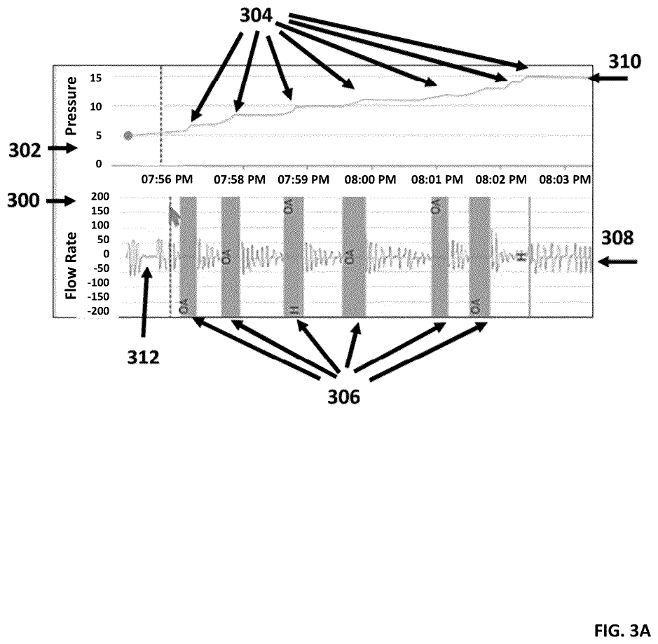

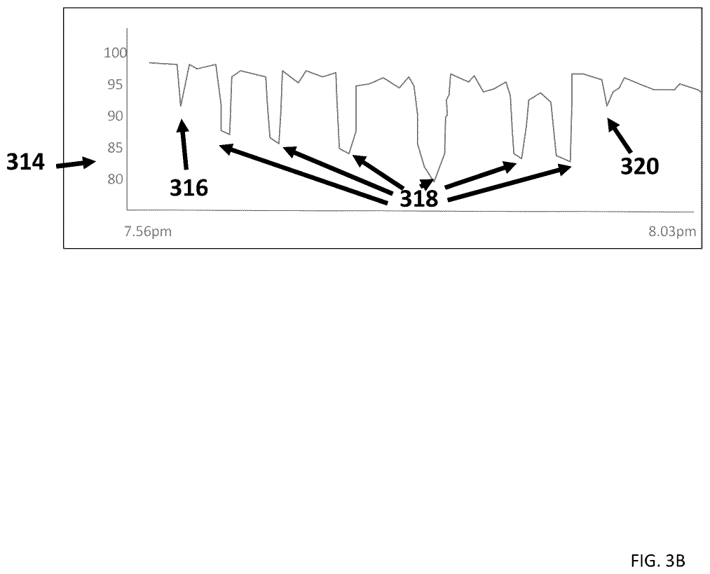

FIG. 3A shows an example of an APAP correctly responding to a series of events. Data plot 300 is a time sequence of air flow into the lungs as detected by an airflow sensor in the APAP mask. The up and down traces indicate inhale and exhale cycles. When the air flow is reduced or is stopped, it is deemed an indication of hypopnea or apnea events respectively. A first apnea event is detected at 312. This does not invoke a pressure change in this case, as more than one event may be detected before an increase in pressure is made to avoid unnecessary pressure changes based on a single event. Pressure changes are shown in data plot 302. However, a sequence of six apnea events follows at 306 over a period of approximately five minutes, which equates to a running value of AHI=60 which is considered high and in need of alleviation. These events trigger an increase in pressure in stages 304, until sufficient airflow increase enables regular breathing to re-establish at 308, whereby the pressure may then be subsequently reduced starting at 310. In this example, the base pressure rate is set at 5 cm, and the maximum at 15 cm. If apnea or hypopnea events re-occur, the process will repeat starting at the pressure value occurring at the time of the re-occurrence. In this case, the device may determine a running value of AHI which represents a satisfactory resolution of the patient's SDB status. Typically, the running AHI is reduced to below 10 and preferably below 5.

FIG. 3B shows values of SpO₂ 314, detected by an independent direct blood oxygen sensor concurrently over the same time period as in FIG. 3A for the same patient. The first drop 316 from 98% to 92% corresponds to a reduction in flow rate. As the first event in this sequence, no change in pressure/flow-rate was invoked. The subsequent six decreases 318 of SpO₂ correspond to six major restrictions in flow rate classed as apnea events with significant decreases in SpO₂ for extended periods until the stepwise increases in pressure/flow-rate eventually open-up the airways and there is return to normal breathing. Detected decreases in SpO₂ usually lag flow rate interruptions as it takes time for oxygen depletion to develop due to a reservoir of oxygen stored in the cardio-vascular system. Furthermore, delays also result when the blood oxygen sensor is placed peripherally, such as on a finger. Delays in oxygen depletion, for example, may take multiple seconds (e.g. 5-15 seconds) after interruptions of the flow rate, and the length of the delays may be influenced by various factors including but not limited to quality of breathing just prior to the interruptions. The small decline in SpO₂ at 320 correspondents to a partial restriction of flow rate in FIG. 3A labeled H indicative of a hypopnea event.

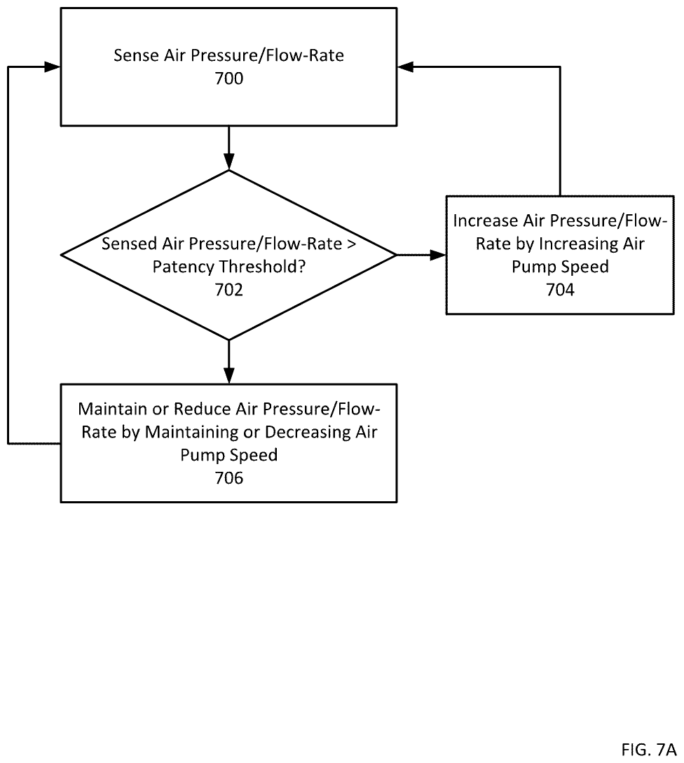

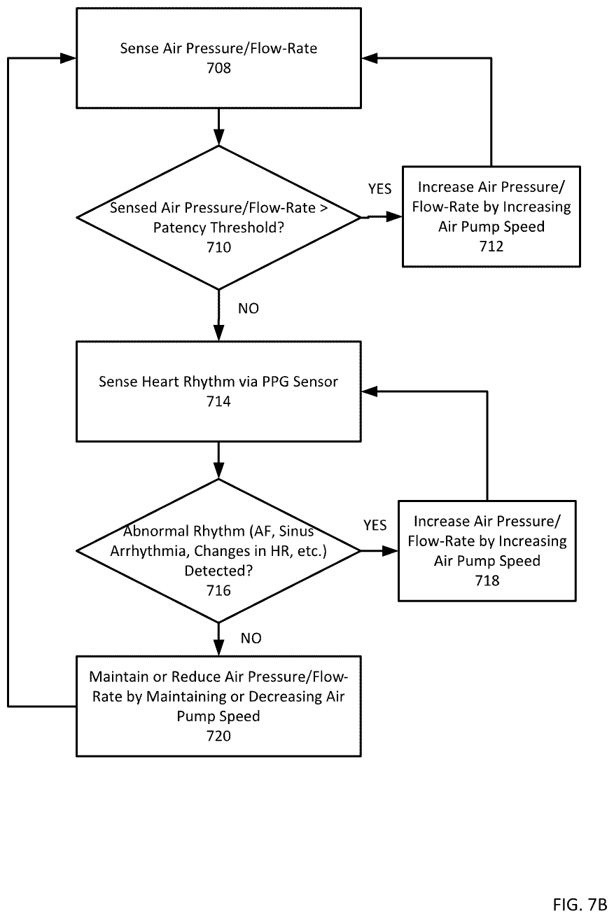

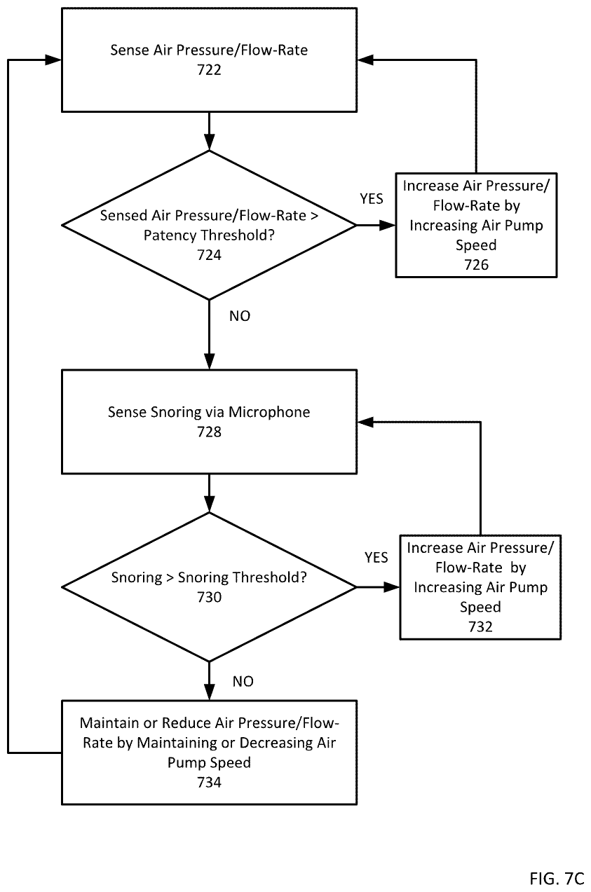

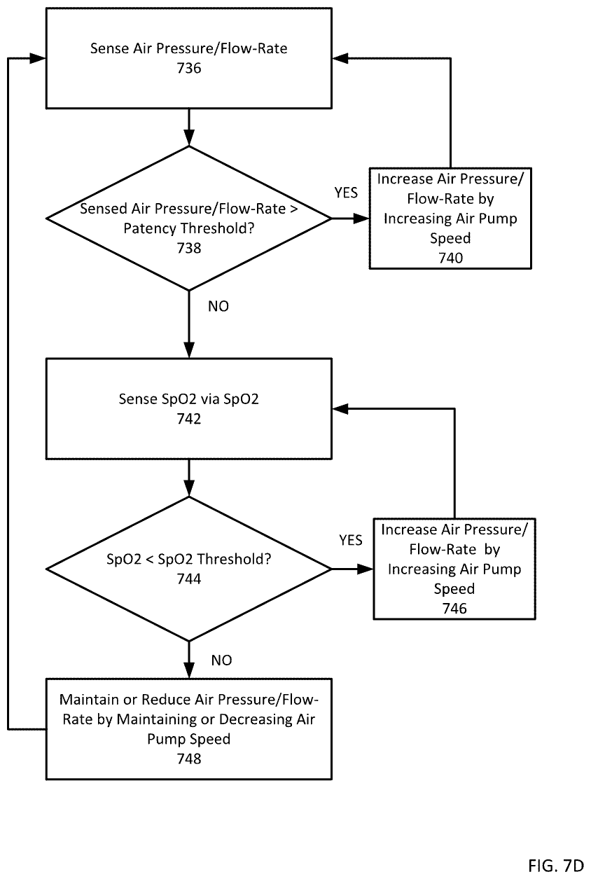

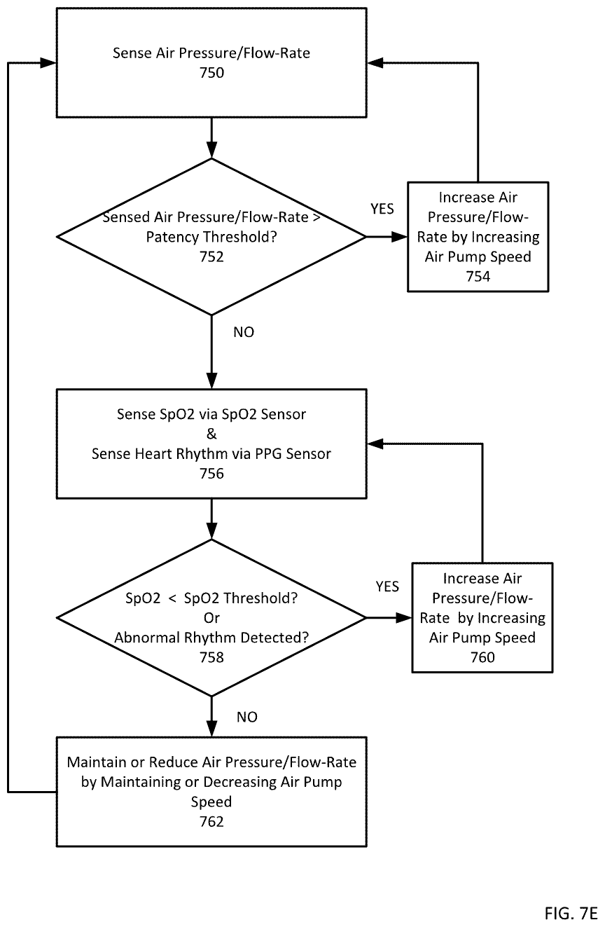

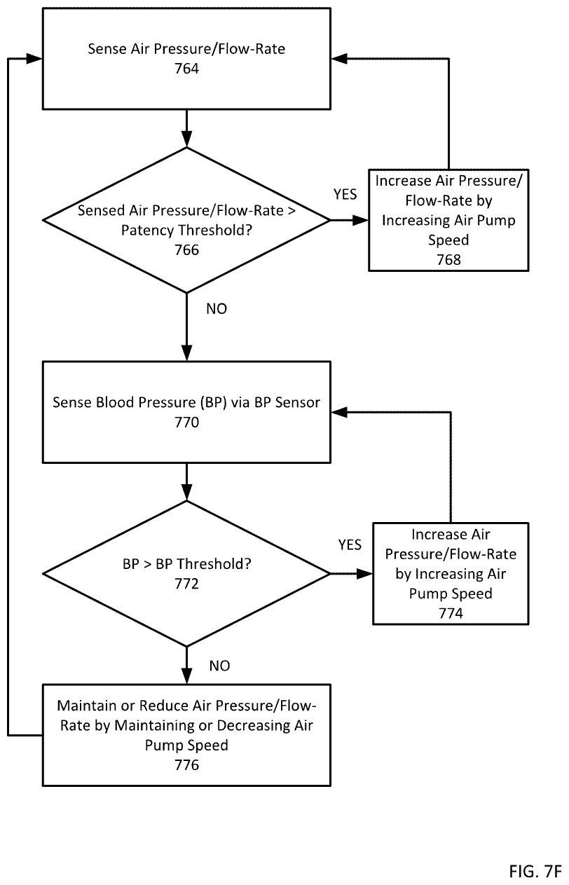

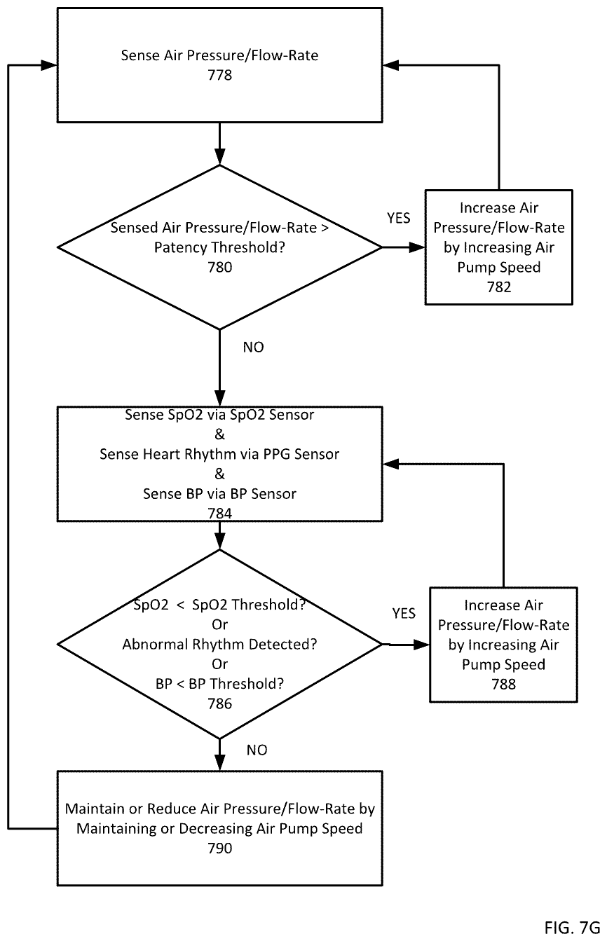

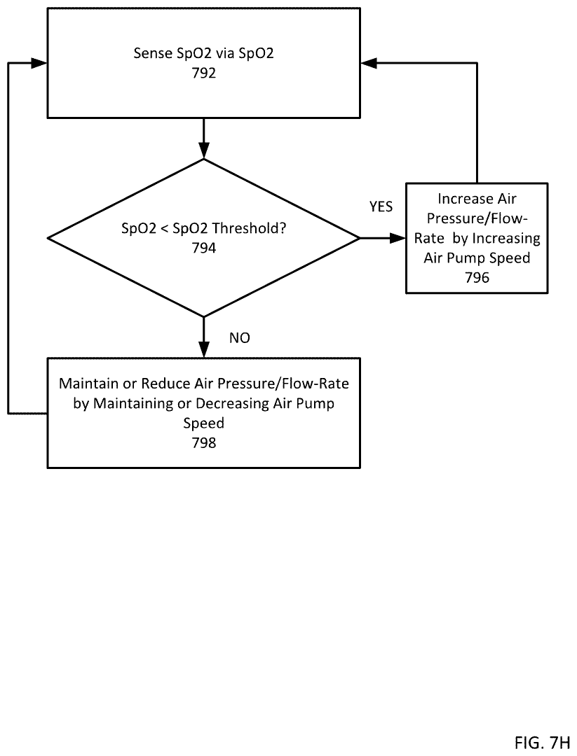

The charts in FIGS. 3A and 3B represent a satisfactory operation of an APAP device whereby detection of flow rate alone, an indirect parameter, results in responses in pressure/flow-rate change. Alternatively, as illustrated in FIGS. 3A and 3B, modification of the required pressure/flow-rate using just the input from the ‘direct’ parameter SpO₂ sensor would also provide a resolution to the observed SDB symptoms. Thus, using just an SpO₂ sensor instead of monitoring pressure/flow-rate would have provided a similar therapeutic benefit. Either sensing method is satisfactory in this example. In one example embodiment, the air-way flow-rate or pressure sensors are replaced with an SpO₂ sensor with or without an HR sensor. This example embodiment may simplify the structure of the APAP and reduce manufacturing costs as illustrated in FIG. 7H.

However, in certain circumstances, the measurement of air pressure/flow-rate as an indirect factor for determination of treatment efficacy may not provide adequate therapy. Using a direct factor (e.g. SpO₂) may be a better parameter for pressure/flow-rate control in circumstances where indirect parameters are insufficient. For example, in some patients, SpO₂ may fall even when there is only a small reduction in the indirect airflow parameter insufficient to be detected by the APAP device, or the event(s) do not reach the restrictions in airflow or meet the pre-determined durations and intensities used to define apnea or hypopnea events. In other words, the PAP device may not detect the airflow restriction and therefore not increase the air pressure/flow-rate even though the patient is suffering from lack of oxygen. Thus, it is possible in such cases that air pressure/flow-rate alone is insufficient to trigger a rise in applied air pressure/flow-rate. For example, there may be a series of flow restrictions that last for nine seconds each, or drop by 45%, or some combination of these two parameters that prevent the events being classified based on the standard definitions of AHI. Nevertheless, the flow restrictions may still be sufficient to cause hypopnea or even apnea events as well as triggering onset of AF as determined by drops in the direct parameter SpO₂ and/or the detection of AF onset. In this case the device may continue to report a low value of running AHI although the patient is experiencing dangerous levels of oxygen desaturation and/or AF, and/or snoring.

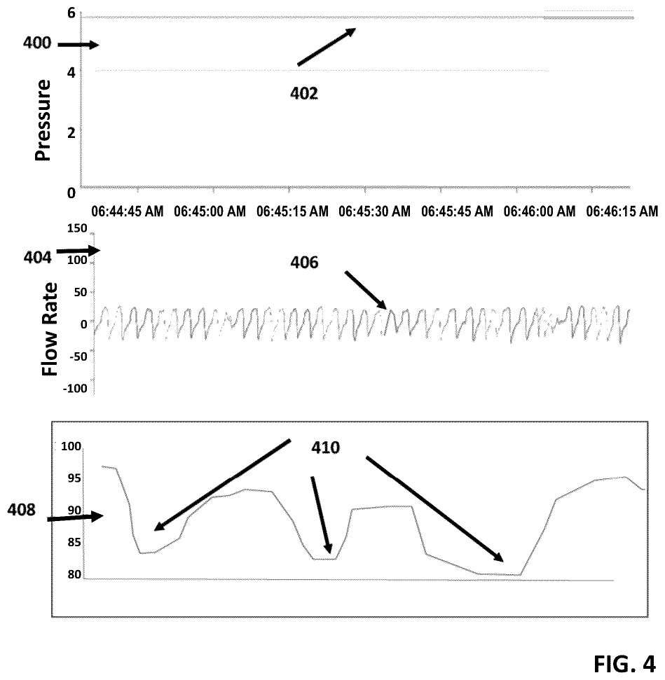

To illustrate this problem in further detail, FIG. 4 shows an example in which indirect airflow sensing is insufficient to invoke an appropriate pressure/flow-rate increase although oxygen desaturation is occurring. The flow rate chart 404 shows stable trace 406 both on inhalation and exhalation. However, there may be flow limitations occurring that are not sufficiently large enough to trigger a pressure/flow-rate increase to the airway. For example, as seen in the pressure trace 400, the pressure level 402 remains at the pre-determined minimum value of 5.5 cm. However, a sequence of desaturation events, 410 are occurring as shown in the SpO₂ trace 408. In this example, a doubling of the applied pressure to 11 cm by physician intervention was sufficient to eliminate the additional occurrences of desaturation events. Thus, using a direct physiological parameter, namely SpO₂, to control the applied pressure would overcome the lack of appropriate response using indirect only airway pressure/flow-rate detection and therefore improve therapeutic performance and potentially reduce short and long-term health problems.

An APAP pressure/flow-rate control system using a direct physiological parameter, namely a heartrate sensor provides another example solution. Heartrate sensors may be lower cost, more reliable and accurate than SpO₂ sensors, particularly with sufferers with dark skin pigment or thick or calloused skin. Heartrate can conveniently be detected for example with a sensor placed on the user's finger, earlobe, forehead or other convenient corporal locations. As the patient's heart beats, the blood volume at the location, for example at a finger, periodically fluctuates in correlation with the subject's heart rate. A heart rate sensor sampling at least 30 times a second records these fluctuations, referred to photoplethysmography (PPG) data, during sleep.

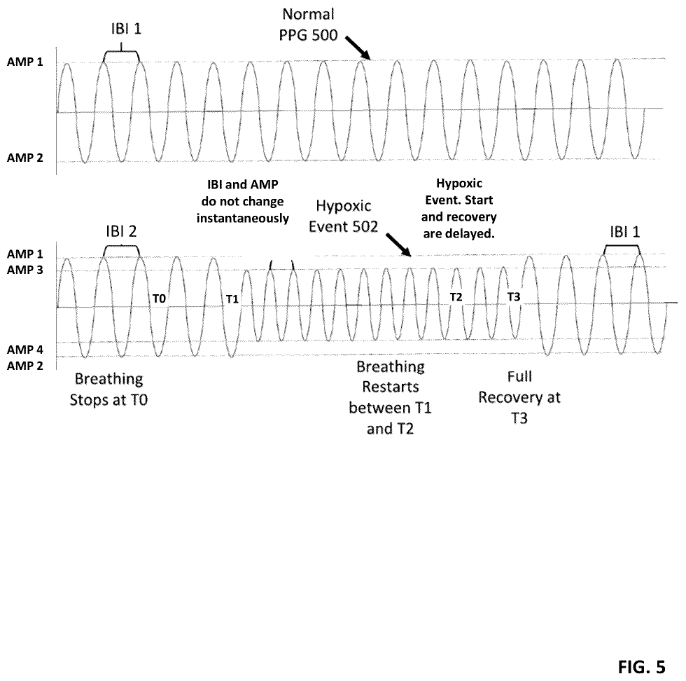

Two examples of typical PPG data captured by a heartrate sensing device are shown in FIG. 5 for illustrative purposes. In the upper chart, a normal PPG data waveform 500 identifies an Interbeat Interval, IBI1, amplitude defined by of AMP1 and AMP2, and a peak-to-peak amplitude of AMP1-AMP2. In general, a normal PPG data waveform 500 includes an IBI and amplitude that is fairly consistent over time assuming the SDB sufferer does not vary their physical activity or breathing during the measurements. IBI1 and AMP1 and AMP2 correlate to adequate blood oxygen levels for the particular subject.

The lower chart illustrates the effect on heartrate and amplitude by a hypoxic event. PPG data waveform 502 starts out with a normal IBI1 and amplitude of AMP1-AMP2 indicating normal blood oxygen levels. However, when the sufferer's breathing is significantly reduced or stopped completely in a hypoxic (or hypopnea event) for example, at time T0, their blood oxygen levels begin to drop which eventually triggers a decrease in IBI2, corresponding to an increase in heartrate as well as in most cases a decrease in AMP3-AMP4 starting at time T1 which correlates to inadequate blood oxygen levels for the particular subject. Although waveform 502 shows the decrease to IBI2 and AMP3 occurring seemingly instantaneously, in practice, these decreases would occur gradually over time after the subject's breathing is significantly reduced or stopped. Once the subject begins breathing again at time T2, their blood oxygen levels begin to rise, eventually triggering an increase back to IBI1 and AMP1 starting at time T3, which again, correlates to adequate blood oxygen levels for the subject. Although waveform 502 shows the increase back to IBI1 and AMP1 occurring seemingly instantaneously, in practice, these increases would also occur gradually over time after the user begins breathing again. Nevertheless, sensing heartrate changes can provide an alternative solution for event detection to SpO₂ sensing.

It is important to note that when using heartrate sensing to detect events, it may be necessary to take into account the possible existence of so-called sinus arrhythmia. Heartrate is known to vary in synchronism with breathing, increasing slightly upon inhale and decreasing slightly upon exhale independent of an SDB event. This regular periodic pattern, referred to as sinus arrhythmia, can be detected by a heartrate sensor and such regular variations may be allowed for when determining whether or not there is an event occurring. Variations in heartrate during inhale and exhale are much smaller than those that are caused by events when there is significant reduction in SpO₂. Typically sinus arrhythmia changes (decreases/increases) the interbeat interval (IBI) of heartrate by around ±0.16 seconds every 4-12 seconds corresponding to a regular breathing pattern, whereas when an event occurs, the IBI decreases by 0.3 second or more and does not follow the regular increase/decrease pattern associated with regular breathing. In order to differentiate between normal sinus arrhythmia and an event, the regular background PPG data established as a baseline is subtracted from the detected PPG trace as in the lower chart of FIG. 5 to remove any artifacts in event detection from background heartrate variations due to sinus arrhythmia. Repetitive cycles of direct heartrate frequency variations with or without amplitude changes as illustrated in FIG. 5 may be used as direct data to control an APAP machine.

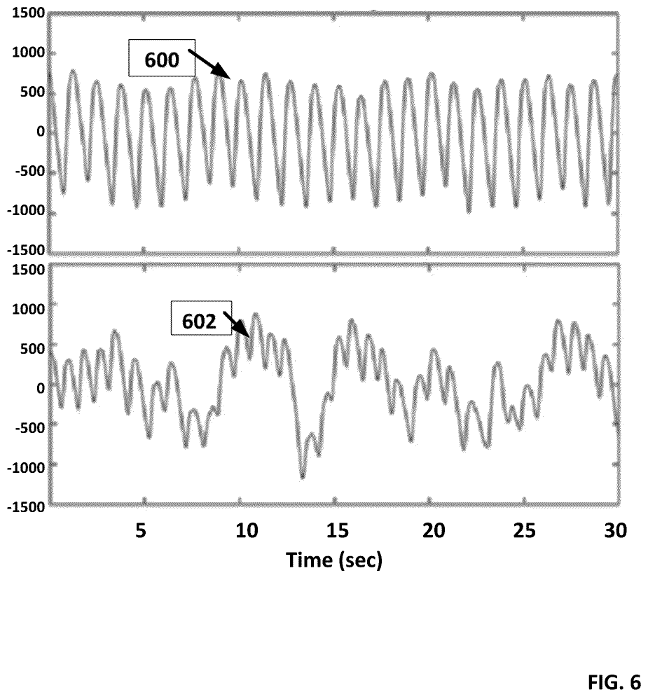

In a PPG signal monitored by a heartrate sensor, AF is manifested as varying pulse-to-pulse intervals, pulse shapes and amplitudes. In contrast, a normal sinus rhythm is recognizable through regularly spaced PPG pulses with similar morphologies between consecutive pulses. FIG. 6 shows a normal PPG trace 600, compared with one where AF is prevalent in trace 602. There are several different algorithms that have been shown to be capable of differentiating between normal and AF PPG traces. For example, a Fast Fourier Transform (FFT) is extracted from running samples of ‘n’ pulses where ‘n’ is at least 10 and preferably at least 20. The derived frequency spectrum (not shown) for a normal PPG trace will show a clearly defined single frequency peak matching the regular heartrate (e.g. between 0.6 to 2.0 Hz). If the subject exhibits strong sinus arrythmia a second clearly defined frequency peak matching the rest breathing rate between 0.16 and 0.33 Hz will be seen. However, when AF is occurring, one of both of these well-defined frequency peaks will be weak or non-existent.

Machine learning (ML) methods using classifiers from multiple records of PPG traces from diagnosed sufferers of AF have been successful in diagnosing AF from PPG traces. Further so-called Deep learning (DL) models, unlike ML automatically learn feature representations from subject's own datasets, sparing the tedious task of feature crafting using ML methods. DL uses a neural network, a set of interconnected layers of computational nodes. The most common DL approaches, used for AF detection, are based on Convolutional Neural Networks (CNN). These methods and others have been shown to be as accurate as ECG diagnosis techniques thus enabling single wearable PPG sensors to substitute more complex multi-electrode ECG methods.

A further direct physiological parameter that may be used to improve the control of APAPs is blood pressure (BP). SDB events as well as occurrence of AF often trigger increases in BP. Wearable technology is now available to periodically check for BP and is expected to provide continuous tracking of BP in the future. For example, a device which uses PPG signal analysis from a single sensor finger wearable may provide a cuffless, calibration-free continuous blood pressure monitor.

Additionally, it is noted that two or more direct parameters such as SpO₂, HR, HV and BP changes can be combined to increase the accuracy when used to control air pressure/flow-rate applied by an APAP, with or without taking into account the indirect parameters derived from airway pressure/flow-rate. Effectively, the processor can perform real-time control of the air pressure/flow-rate during sleep sessions by switching between controlling the air pressure/flow-rate based on indirect and/or direct parameters as needed.

FIGS. 7A-7H illustrate how APAP pressure/flow-rate can be controlled in real-time during sleep sessions when one, two, three or more sensors including direct physiological parameter sensors monitoring physiological parameters and indirect pressure/flow-rate sensors monitoring airway pressure/flow-rate. The physiological parameters may be compared to physiological parameter thresholds, whereas the airway pressure/flow-rate sensors may be compared to pressure/flow-rate thresholds. Multiple sensing provides additional safety and improved therapeutic outcomes.

For example, FIG. 7A shows a flowchart of APAP pressure/flow-rate control using only indirect airway patency sensing (i.e. an indirect method). Air pressure/flow-rate can be measured by a pressure/flow-rate sensor in the PAP device. Alternatively, air pressure/flow-rate can be determined by measuring the electric current used by an air pump which may be a fan, a piezo-electric pump, a reciprocating linear motor, or the like for generating (i.e. producing) an air pressure/flow-rate by blowing air into the PAP mask of the user. The air pressure/flow-rate values are used as thresholds to trigger an increase in pressure. There may be several pre-set values which trigger different amounts of air pressure/flow-rate increases as pre-set by a sleep physician. FIG. 1A, for example, shows how such a configuration may increase the pressure until a satisfactory pressure is achieve. The general control flow shown in FIG. 7A is that the PAP device senses air pressure/flow-rate in step 700 and determines in step 702 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 704. If the sensed air pressure/flow-rate is not greater than the threshold (i.e. the user's airway is open), then the PAP device maintains or reduces the air pressure/flow-rate by maintaining/reducing the air pump speed in step 706.

FIG. 7B shows a flowchart of APAP pressure/flow-rate control using a combination of indirect airway patency sensing and direct sensing of heart rhythm. The general control flow shown in FIG. 7B is that the PAP device senses air pressure/flow-rate in step 708 and determines in step 710 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 712. If the sensed air pressure/flow-rate is not greater than the threshold (i.e. the user's airway is open), then the PAP device senses heart rhythm in step 714 and determines if an abnormal rhythm (i.e. pattern) such as Atrial Fibrillation, Abnormal Sinus Arrythmia, Tachycardia, or any rhythm that is determined to be abnormal is detected in step 716. Heart rhythm can be detected, for example, by a PPG sensor or the like placed against the user's skin. If an abnormal heart rhythm is detected, then the PAP device increases air pressure/flow-rate by increasing air pump speed in step 718. If the heart rhythm is normal, then the PAP device maintains or reduces the air pressure/flow-rate by maintaining/reducing the air pump speed in step 720.

It is noted that the PPG sensor provides for the continuous tracking of any heart rate abnormality including the onset of AF, whereupon control of the APAP pressure occurs in order to alleviate AF symptoms. The PPG data can also be used to derive background sinus arrhythmia data to be used to correct event detection by removing background sinus arrhythmia fluctuations present in the heartrate. An OSA event using PPG data, for example may be defined by a combination of a sudden increase of heartrate by at least 10% from a baseline value, followed by a return to the baseline value with an interval of a preset time in seconds between 10 and 30 seconds. For example, a typical baseline heartrate during sleep may be 70 bpm. When heartrate increase to 77 bpm time zero of an event is recorded. If and when the heartrate returns to 70 BPM, the event end time is again recorded. If the time is between 10 and 30 seconds, a SDB event is deemed to have occurred. In practice, a single such event does not create a change in PAP pressure. However, two or more events occurring within between 1 and 5 minutes may invoke a pressure increase even if it had not been triggered by the defined airway patency threshold. Once events no longer occur, the pressure is reduced.

FIG. 7C shows a flowchart of APAP pressure/flow-rate control using a combination of indirect airway patency sensing and direct sensing of snoring. The general control flow shown in FIG. 7C is that the PAP device senses air pressure/flow-rate in step 722, and determines in step 724 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 726. If the sensed air pressure/flow-rate is not greater than the threshold (i.e. the user's airway is open), then the PAP device senses snoring in step 728 and determines if snoring is greater than a threshold in step 730. Snoring can be detected, for example, by a microphone, a vibration sensor, or the like placed on the mask, the PAP machine or in proximity to the user (e.g. smartphone microphone). The snoring threshold may be a based on at least one of intensity and duration of the snoring. If snoring is greater than the snoring threshold (e.g. sound volume, vibration intensity, frequency, and/or duration), then the PAP device increases air pressure/flow-rate by increasing air pump speed in step 732. If the snoring is not greater than the snoring threshold, then the PAP device maintains or reduces the air pressure/flow-rate by maintaining/reducing the air pump speed in step 734.

It is noted that the sound sensor provides for the continuous tracking of any onset of SE's whereupon control of the APAP pressure occurs in order to alleviate snoring. This configuration is particularly suitable for a patient known to suffer from snoring during sleep and the PAP is being used to alleviate these symptoms. Snoring indices are derived from level and frequency of events derived after processing of the raw audible signals as described above. For example, snoring indices may be classified as mild, medium and intense. At the mild level, the control algorithms may be programmed to not increase airway pressure. At the medium level, the pressure is increased, for example, in steps of 10% for one minute until the snoring is eliminated. If this is insufficient, the pressure is increased to the maximum value set in the PAP. At intense level snoring, pressure is increased, for example, by 20% every minute to the maximum allowed. When snoring ceases, the applied pressure is slowly reduced to the present minimum unless snoring recommences whereupon the increasing protocols are repeated.

FIG. 7D shows a flowchart of APAP pressure/flow-rate control using a combination of indirect airway patency sensing and direct sensing of SpO2. The general control flow shown in FIG. 7D is that the PAP device senses air pressure/flow-rate in step 736, and determines in step 738 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 740. If the sensed air pressure/flow-rate is not greater than the threshold (i.e. the user's airway is open), then the PAP device senses SpO2 in step 742 and determines if SpO2 less than a threshold (e.g. threshold indicating low SpO2) in step 744. SpO2 can be detected, for example, by an SpO2 sensor placed in contact with the user's skin. If the SpO2 is less than the SpO2 threshold, then the PAP device increases air pressure/flow-rate by increasing air pump speed in step 746. If the SpO2 is not less than the SpO2 threshold, then the PAP device maintains or reduces the air pressure/flow-rate by maintaining/reducing the air pump speed in step 748.

FIG. 7E shows a flowchart of APAP pressure/flow-rate control using a combination of indirect airway patency sensing and direct sensing of both heart rhythm and SpO2. The general control flow shown in FIG. 7E is that the PAP device senses air pressure/flow-rate in step 750, and determines in step 752 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 754. If the sensed air pressure/flow-rate is not greater than the threshold (i.e. the user's airway is open), then the PAP device senses Heart Rhythm and SpO2 in step 756 and determines if an abnormal heart rhythm is detected or if SpO2 is less than the SpO2 threshold in step 758. If an abnormal heart rhythm is detected or if SpO2 is less than the SpO2 threshold, then the PAP device increases air pressure/flow-rate by increasing air pump speed in step 760. If the heart rhythm is normal and the SpO2 is not less than the SpO2 threshold, then the PAP device maintains or reduces the air pressure/flow-rate by maintaining/reducing the air pump speed in step 762.

FIG. 7F shows a flowchart of APAP pressure/flow-rate control using a combination of indirect airway patency sensing and direct sensing of blood pressure. The general control flow shown in FIG. 7F is that the PAP device senses air pressure/flow-rate in step 764, and determines in step 766 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 768. If the sensed air pressure/flow-rate is not greater than the threshold (i.e. the user's airway is open), then the PAP device senses blood pressure (BP) in step 770 and determines if BP is greater than a threshold (e.g. threshold indicating high BP) in step 772. BP can be detected, for example, by an BP sensor placed in contact with the user's skin. If the BP is greater than the BP threshold, then the PAP device increases air pressure/flow-rate by increasing air pump speed in step 774. If the BP is not greater than the BP threshold, then the PAP device maintains or reduces the air pressure/flow-rate by maintaining/reducing the air pump speed in step 776.

FIG. 7G shows a flowchart of APAP pressure/flow-rate control using a combination of indirect airway patency sensing and direct sensing of heart rhythm, SpO2 and blood pressure. The general control flow shown in FIG. 7G is that the PAP device senses air pressure/flow-rate in step 778, and determines in step 780 if the sensed air pressure/flow-rate is greater than a patency threshold (e.g. threshold indicating if the user's airway is obstructed). If the sensed air pressure/flow-rate is greater than the threshold (i.e. the user's airway is obstructed), then the PAP device increases air pressure/flow-rate by increasing air pump speed in step 782. If the sensed air pressure/flow-rate is not greater than the threshold (i.e. the user's airway is open), then the PAP device senses Heart Rhythm, BP and SpO2 in step 784 and determines if an abnormal heart rhythm is detected, or if BP is greater than the BP threshold, or if SpO2 is less than the SpO2 threshold in step 786. If an abnormal heart rhythm is detected, or if BP is greater than the BP threshold, or if SpO2 is less than the SpO2 threshold, then the PAP device increases air pressure/flow-rate by increasing air pump speed in step 788. If the heart rhythm is normal and BP is not greater than the BP threshold, and SpO2 is not less than the SpO2 threshold, then the PAP device maintains or reduces the air pressure/flow-rate by maintaining/reducing the air pump speed in step 790.

FIG. 7H shows a flowchart of APAP pressure/flow-rate control using direct sensing of SpO2. FIG. 7H is different than FIGS. 7B-7F in that the PAP device does not sense air pressure/flow-rate (i.e. the SpO2 sensor replaces the pressure/flow-rate sensor). Instead, the PAP device senses Sp02 in step 792 and determines if SpO2 less than a threshold (e.g. threshold indicating low SpO2) in step 794. If the SpO2 is less than the SpO2 threshold, then the PAP device increases air pressure/flow-rate by increasing air pump speed in step 796. If the SpO2 is not less than the SpO2 threshold, then the PAP device maintains or reduces the air pressure/flow-rate by maintaining/reducing the air pump speed in step 798. It is noted that SpO2 can be detected, for example, by an SpO2 sensor placed in contact with the user's skin. It is noted that SpO2 detection is more beneficial and reliable than indirect airway patency. Therefore, the SpO2 sensor can be substituted for the air pressure/flow-rate sensor in order to offset manufacturing costs.

The decision flow-charts shown in FIGS. 7A-7H are just some examples of how APAP pressure control can be improved by introducing one or more direct physiological sensors into the control algorithms. Other control parameter combinations can also be envisioned. For example, any combination of one or more of airway patency, HR rhythm, BP, SpO2 and the like can be used to control the air pump of the APAP to increase, decrease and maintain the pressure/flow-rate when beneficial to the user. It should also be noted that the monitoring steps in FIGS. 7A-7H may be performed continuously or periodically, and that the increase in air pressure/flow-rate may be performed immediately when the monitored parameters fall below their respective thresholds, or the increase in air pressure/flow-rate may be performed when the monitored parameters fall below and stay their respective thresholds for a predetermined period of time in order, for example, to avoid false positive indications of SBD events.

In addition, as other such sensors become available, decision trees may be modified/created to take into account their accuracy, response time and other sensor features. Furthermore, the sensed data for multiple sleep sessions and analytical algorithms can be stored and executed either within a wearable sensing device such as a ring, a sensing device within the PAP mask, within the PAP device, standalone sensing devices in proximity to the user, or a combination of these sensing devices.

Such sensors can be positioned at a number of different locations such as:
On an ear-lobe. This location has the advantage that it can be connected directly to APAP mask. For example, the sensor may be housed within a device that has other functions such as an ear-bud or hearing-aid.
Inside an ear canal. For example, the sensor may be housed within a device that has other functions such as an ear-bud or hearing-aid.
On a wrist, or ankle or finger-tip or toe. These locations provide comfort and reliable placement.
On or near the face.

Such sensors may be hard-wired to the PAP device, or preferably use wireless data communication interfaces and methods such as, but not limited to, Bluetooth™ protocols for communicating data and/or instructions to the PAP device.

Suitable blood oxygen concentration sensors sample values of SpO₂ at rates sufficient to enable the pressure/flow-rate control algorithms to function in a timely manner when oxygen desaturation levels demand a change in applied pressure/flow-rate. A sampling rate, for example, of least twenty times a second or more could be used with an accuracy per sample of +/−1% of the detected value.

Suitable heartrate sensors should sample pulse rate at rates sufficient to enable the pressure/flow-rate control algorithms to function in a timely manner when heartrate patterns demand a change in applied pressure/flow-rate. A sampling rate, for example, of least fifty times a second may be employed with an accuracy per sample of +/−1% of the detected value. Whether a pressure/flow-rate change is invoked depends on the detected desaturation and/or heartrate data patterns. A single isolated desaturation event may not invoke a pressure/flow-rate change. When a number of events occur in a defined time period (e.g. five minutes) s the applied pressure/flow-rate is modified. These metrics can be defined by a set of parameters which may be pre-set by the manufacturers of the APAP device, or alternatively, may be defined by a sleep physician to meet the specific SDB symptoms of an individual patient.

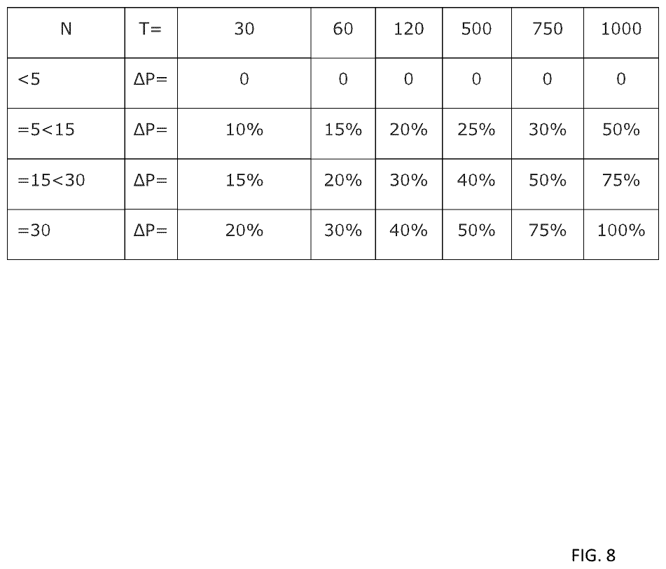

FIG. 8 is one example of an increase in pressure decision matrix based on monitored SpO₂ data. For example, in FIG. 8, N equals the number of desaturation events per minute in which SpO₂ drops by more than 3% from a mean value, and T is the time in seconds over which a sequence of such 3% reductions have occurred. ΔP is the percentage increase in pressure from a mean baseline value determined prior to any 3% desaturation event. If the first stage of pressure increase reduces the number of 3% desaturation events, then further pressure increases are not applied unless or until further such events occur.

A similar decision matrix (not shown) can be implemented based on heartrate change data used to detect an event where N equals the number of events per minute in which heartrate increases by more than 10% from a mean value over a period in the range fifteen to sixty seconds, and T is the time in seconds over which a sequence of such 10% increases have occurred. In this example, ΔP is the percentage increase in pressure from a mean baseline value determined prior to any heartrate increase event. If the first stage of pressure increase reduces the number of 10% short-term heartrate increase events, then further pressure increases are not applied unless or until further such events occur.

Once the apnea or hypopnea events are resolved, the pressure is reduced gradually to the baseline value unless events are again detected at which time pressure increase protocols are invoked starting at the prevailing value. Such decision matrices may be modified to personalize pressure changes to match an individual's air flow and/or desaturation patterns and/or heartrate increases over time by a physician and/or using machine learning methods.

It is noted that all relevant data may be securely encoded and uploaded to a central secure database via the internet for algorithmic processing to determine appropriate pressure control commands which are communicated to the PAP device and to undertake analyses (e.g. ML or DL) over time to modify decision matrices for individual patients and predict dangerous events prior to severe onset of symptoms. Similar methods can be used for other combinations of direct sensors. This configuration has a further benefit, which is the ability to correlate the timing and intensity of events and AF episodes is valuable to healthcare professionals when deciding on which therapies are most appropriate for alleviating AF. Sleep triggered AF is only one of several underlying causes of AF. Further, the onset of AF by an event usually occurs at the end of the event when the central nervous system is most aroused. By recording and storing sensed data for both events and onsets of AF over time, for example, 100 co-occurring events and related AF episodes, such data can be analyzed using machine learning (ML) or deep leaning (DL) algorithms to predict when an AF episode is likely to be triggered and make adjustments to the APAP pressure prior to onset and hence reduce the likelihood of dangerous AF onset.

As discussed above, the disclosure ensures that appropriate pressure/flow-rate changes are made by an APAP device in cases whereby reductions in air flow and/or airway pressure/flow-rate are not sufficient to invoke a pressure/flow-rate increase or decrease, and/or the patient is experiencing one or more oxygen desaturations and/or periodic variations in heartrate and heartrate amplitude, that have not been addressed sufficiently by the existing pressure/flow-rate control algorithms, or there is a malfunction in the device or the patient is suffering from issues that do not necessarily result in airway flow restrictions such as AF or SE.

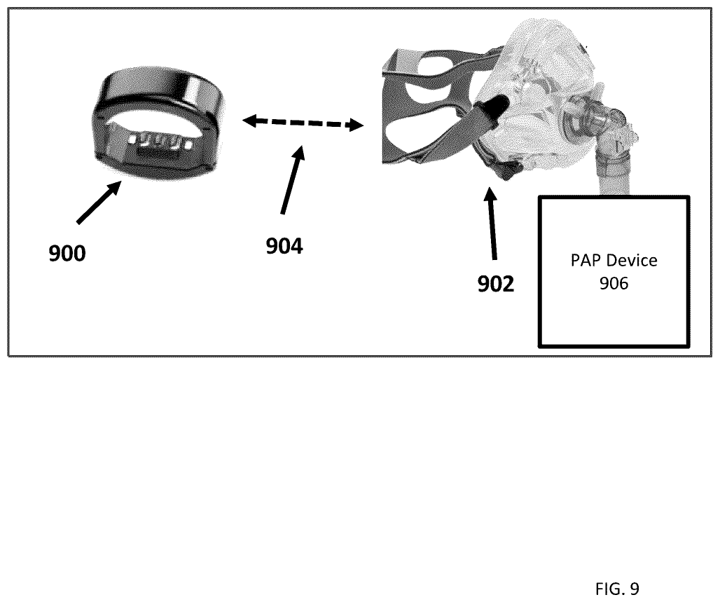

FIG. 9 shows an example of a ring 900 worn by the patient during sleep that has at least a continuous blood oxygen sensor and a heartrate sensor suitable for use in the flowcharts described above. For example, the sensor samples SpO₂ at a defined rate, for example, at least 20 times a second with a relative accuracy of +/−1% and samples at a defined rate, for example, pulse amplitude at least 100 times a second with a relative accuracy of +/−1%. The ring 900 communicates using low power Bluetooth™ protocols via wireless link 904 to an electronic control module (not shown) within an APAP mask 902 or within the APAP device 906. Examples of such rings are Circul+ from Bodimetrics, the Belun®Ring from BelunTech, the GotoSleep ring from SleepOn, among others. Manufacturers of APAP devices include Fisher & Paykel, Resmed, and Respironics, a division of Philips, among others.

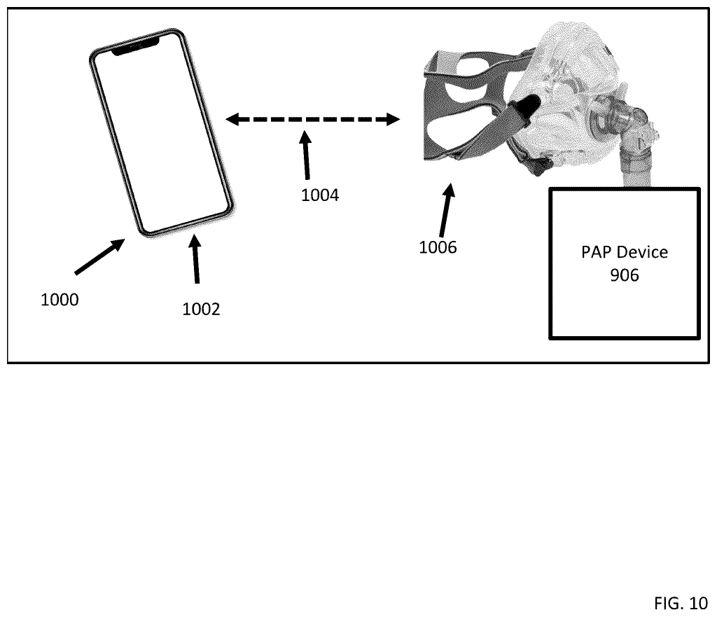

FIG. 10 shows an example embodiment suitable for detection and alleviation of snoring episodes in which sounds are detected by the microphone 1002 within a smart phone 1000. After the raw sound signals are processed to remove background noise, and algorithms determine intensity of the occurrence of snoring. The smart phone 1000 then transmits instructions via link 1004 to the PAP device 906 to increase airway pressure/flow-rate until the snoring is diminished.

In other examples, snoring and other physiological parameters can be detected using a microphone, vibration sensors or other sensors (not shown) embedded in or physically attached to the PAP device 906 or mask 1006. In the case of snoring, such a configuration may be beneficial to reduce the impact of unwanted external ambient sounds, sounds from sleep partners, and unrelated sounds from patient movements.

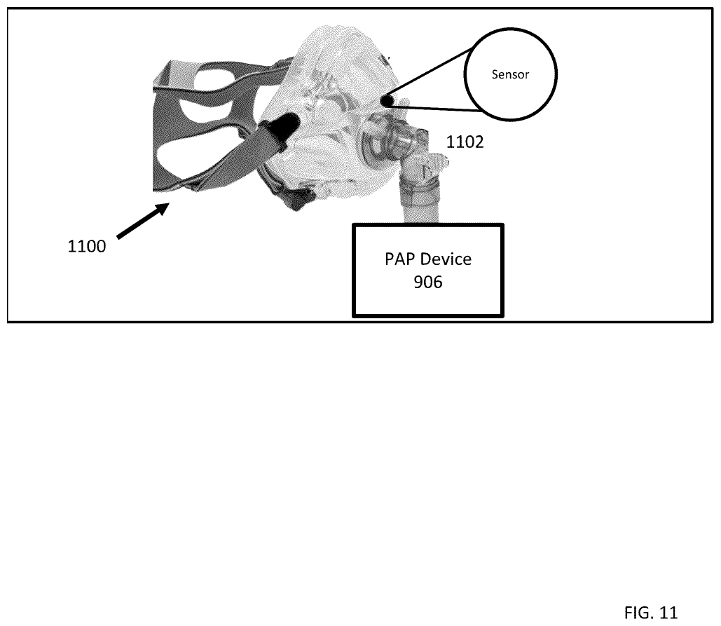

For example, FIG. 11 shows an example embodiment where a sensor 1102 is attached to or embedded (i.e. integrated) into a PAP mask 1100. The sensor may be an SpO2 sensor, HR sensor, BP sensor, microphone, vibration sensor or the like for sensing of direct user parameters. For example, the sensor may be microphone or vibration sensor for detecting SE. After the raw sound signals are received and processed (e.g. by the smartphone or the PAP device itself) to remove background noise, the algorithms determine intensity of the occurrence of snoring, and the PAP controller is directed to increase airway pressure/flow-rate until the snoring is diminished.

It is noted that other sensors may also be used for controlling the APAP. For example, movement sensors (e.g. accelerometer, video camera, radar transceiver, etc.) may be used to detect user movement (e.g. breathing patterns, other movements correlated to SDB) during sleep. In one example, an accelerometer can be worn by the user on their diaphragm or stomach as a wearable device. In another less invasive example, a video camera or radar transceiver may be positioned in the user's bedroom (e.g. on the nightstand) and may capture video or radar distance measurements to user as they sleep. Analysis of the captured video or radar distance measurements may indicate user movement (e.g. breathing patterns, other movements correlated to SDB) during their sleep. In one example, the APAP may adjust pressure/flow-rate if the movement sensor detects a change in breathing patterns of the user (e.g. user breathing pattern changes from a normal baseline breathing pattern to a breathing pattern indicating that the user is struggling for air). In addition, it is noted that the above-described movement sensors could also be used in conjunction with other sensors such as the wearable ring that detects SpO₂ and/or HR of the user to ensure more accurate control of the APAP pressure/flow-rate.

Algorithms may analyze one or both the heartrate and SpO₂ data, with or without in combination with concurrent sensed flow rate and/or airway resistance data, and using a decision matrix that employs desaturation event patterns derived from either or both SpO₂ and heartrate time series data, instructs the pressure/flow-rate controller in the APAP device when and at what pressure/flow-rate level to apply airway pressure/flow-rate changes and when to maintain pressure/flow-rate levels. The sensed data for multiple sleep sessions and analytical algorithms can be stored and executed either within a sensing devices such as a ring, or within the APAP device, or shared between the two devices. Alternatively, all relevant data may be securely encoded and uploaded to a central secure database via the internet for algorithmic processing to determine appropriate pressure/flow-rate control commands which are communicated to the APAP device and to undertake ML of DL analyses over time to modify decision matrices for individual patients and predict dangerous events prior to severe onset of symptoms.

It is also noted that the processing of the sensor data and decision making can be performed by one or more of the PAP device, smartphone or backend server. In one example, the PAP device may be the sole processing device that receives the sensor data, processes the sensor data and controls pressure/flow-rate based on the processed sensor data. In another example, the PAP device may work in conjunction with a smartphone and/or backend server. For example, a software application running on the smartphone may collect sensor data, process the sensor data (with or without the aid of a backend server) and then send instructions to the PAP device for controlling pressure/flow-rate.

While the foregoing is directed to example embodiments described herein, other and further example embodiments may be devised without departing from the basic scope thereof. For example, aspects of the present disclosure may be implemented in hardware or software or a combination of hardware and software (e.g. processors, memory devices, electronic circuits, and the like of the PAP device, sensors, smartphone, servers, etc.). One example embodiment described herein may be implemented as a program product for use with a computer system. The program(s) of the program product define functions of the example embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed example embodiments, are example embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

1. A positive airway pressure (PAP) device, comprising:
an air pump for blowing air into a mask worn by a user;
a sensor for detecting air pressure or air flow-rate produced by the air pump;
a communication interface for communicating with a physiological parameter sensor worn by the user for detecting a physiological parameter of the user; and
a processor configured to:
control the air pump to blow the air into the mask according to the air pressure or air flow-rate sensed by the sensor to achieve a prescribed air pressure or air flow-rate, and
control the air pump to adjust the prescribed air pressure or air flow-rate in response to the detected physiological parameter.

2. The PAP device of claim 1, wherein the PAP device is one of a continuous positive airway pressure (CPAP) device, an automatic positive airway pressure (APAP) device, or a Bi-Level positive airway pressure (BiPAP) device.

3. The PAP device of claim 1, wherein the physiological parameter sensor is worn by the user separate from the mask.

4. The PAP device of claim 1, wherein the physiological parameter sensor is integrated into the mask or the PAP device.

5. The PAP device of claim 1, wherein the physiological parameter sensor is a blood oxygen sensor and the processor is configured to:
monitor blood oxygen levels of the user over time, and
control the air pump to increase the prescribed air pressure or air flow-rate in response to the blood oxygen levels decreasing below a blood oxygen level threshold.

6. The PAP device of claim 1, wherein the physiological parameter sensor is a heart rate sensor and the processor is configured to:
monitor heart rate patterns of the user over time, and
control the air pump to increase the prescribed air pressure or air flow-rate in response to
determining that the heart rate patterns are abnormal.

7. The PAP device of claim 1, wherein the physiological parameter sensor is a blood pressure sensor and the processor is configured to:
monitor blood pressure of the user over time, and
control the air pump to increase the prescribed air pressure or air flow-rate in response to the blood pressure increasing above a blood pressure threshold.

8. The PAP device of claim 1, wherein the processor is configured to control the air pump to adjust the prescribed air pressure or air flow-rate in response to a pressure event indicated by the pressure sensor, or a physiological event indicated by the detected physiological parameter.

9. The PAP device of claim 1, wherein the processor is configured to control the air pump to adjust the prescribed air pressure or air flow-rate in response to the detected physiological parameter being compared to a physiological parameter threshold or a baseline of the user.

10. The PAP device of claim 1, wherein the processor is configured to switch between controlling the air pump based on the detected air pressure or air flow-rate and the detected physiological parameter.

11. A positive airway pressure (PAP) device, comprising:
an air pump for blowing air into a mask worn by a user;
a communication interface for communicating with a physiological parameter sensor worn by the user for detecting a physiological parameter of the user; and
a processor configured to control the air pump to adjust a prescribed air pressure or air flow-rate in response to the detected physiological parameter.

12. The PAP device of claim 11, wherein the physiological parameter sensor is worn by the user separate from the mask.

13. The PAP device of claim 11, wherein the physiological parameter sensor is integrated into the mask or the PAP device.

14. The PAP device of claim 11, wherein the physiological parameter sensor is a blood oxygen sensor and the processor is configured to:
monitor blood oxygen levels of the user over time, and
control the air pump to increase the prescribed air pressure or air flow-rate in response to the blood oxygen levels decreasing below a blood oxygen level threshold.

15. The PAP device of claim 11, wherein the physiological parameter sensor is a heart rate sensor and the processor is configured to:
monitor heart rate patterns of the user over time, and
control the air pump to increase the prescribed air pressure or air flow-rate in response to determining that the heart rate patterns are abnormal.

16. A method for controlling a positive airway pressure (PAP) device, the method comprising:
blowing air, by an air pump, into a mask worn by a user;
communicating, by a communication interface, with a physiological parameter sensor worn by the user for detecting a physiological parameter of the user; and
controlling, by a processor, the air pump to adjust a prescribed air pressure or air flow-rate in response to the detected physiological parameter.

17. The method of claim 16, further comprising:
monitoring, by the physiological parameter sensor, blood oxygen levels of the user over time; and
controlling, by the processor, the air pump to increase the prescribed air pressure or air flow-rate in response to the blood oxygen levels decreasing below a blood oxygen level threshold.

18. The method of claim 16, further comprising:
monitoring, by the sensor, heart rate patterns of the user over time, and
controlling, by the processor, the air pump to increase the prescribed air pressure or air flow-rate in response to determining that the heart rate patterns are abnormal.

19. The method of claim 16, further comprising:
increasing, by the processor, the prescribed air pressure or air flow-rate in response to the detected physiological parameter indicating onset of at least one of a heart rate abnormality, a low SpO2, or snoring.

20. The method of claim 16, further comprising:
maintaining or decreasing, by the processor, the prescribed air pressure or air flow-rate in response to the detected physiological parameter indicating an absence of at least one of a heart rate abnormality, a low SpO2, or snoring.
